(19) United States
(12) Patent Application Publication
(10) Pub. No.: US 2025/0082207 A1
(43) Pub. Date: Mar. 13, 2025

(54) CLASSIFYING TENSIVE STATES USING PHOTOACOUSTIC SIGNALS

(71) Applicant: QUALCOMM Incorporated, San Diego, CA (US)

(72) Inventors: Shounak Uday GORE, Hyderabad (IN); Emily Kathryn BROOKS, Amherst, NY (US); John Keith SCHNEIDER, Williamsville, NY (US); Ye ZHAN, Buffalo, NY (US); Evan Michael BRELOFF, Kenmore, NY (US); Vivekananda Parampalli ADIGA, Williamsville, NY (US)

(21) Appl. No.: 18/463,352

(22) Filed: Sep. 8, 2023

(51) Int. Cl. A61B 5/00; A61B 5/021

(52) U.S. Cl. CPC A61B 5/0095; A61B 5/02108; A61B 5/7264; A61B 5/681

(57) ABSTRACT

An apparatus and associated method may include a light source system that includes a light-emitting component, and a receiver system configured to detect an acoustic wave that corresponds to a photoacoustic response of a blood vessel to light emitted by the light source system. A control system may be configured to determine a wave characteristic from the acoustic wave and to estimate a tensive state classification based on the determined wave characteristic. The tensive state classification may be one of a plurality of tensive state classifications that each include different ranges of diastolic and systolic values.

### TECHNICAL FIELD

This disclosure relates generally to photoacoustic devices and systems.

### DESCRIPTION OF RELATED TECHNOLOGY

A variety of different sensing technologies and algorithms are being implemented in devices for various biometric and biomedical applications, including health and wellness monitoring. This push is partly a result of the limitations in the usability of traditional measuring devices for continuous, noninvasive, and ambulatory monitoring. Some such devices are, or include photoacoustic devices. Although some previously-deployed photoacoustic devices and systems can provide acceptable results, improved photoacoustic devices and systems would be desirable.

### SUMMARY

The systems, methods, and devices of this disclosure each have several aspects, no single one of which is solely responsible for the desirable attributes disclosed herein.

One innovative aspect of the subject matter described in this disclosure can be implemented in an apparatus. The apparatus may include a light source system that includes a light-emitting component, and a receiver system configured to detect an acoustic wave that corresponds to a photoacoustic response of a blood vessel to light emitted by the light source system. A control system may be configured to determine a wave characteristic from the acoustic wave and to estimate a tensive state classification based on the determined wave characteristic. The tensive state classification may be one of a plurality of tensive state classifications that each include different ranges of diastolic and systolic values.

According to some implementations, the control system is further configured to determine a blood pressure reading based on the tensive state classification. In another example, the control system is further configured, based on the tensive state classification, to estimate at least one of: demographic data, activity data, and real-time health data.

In some implementations, the control system is further configured to perform modelling operations on the acoustic wave to estimate a blood pressure reading. In one instance, the control system is further configured to select a predictive modelling operation from a plurality of predictive modelling operations based on at least one of the tensive state classification and the acoustic wave. In another or the same example, the control system is further configured to mathematically weight the wave characteristic to select a modelling operation from among a plurality of predictive modelling operations.

According to some implementations, the control system is further configured to access aggregated data from a database. The aggregated data includes data from a plurality of users and at least one of their associated tensive state classifications and wave characteristics. The control system of an implementation may further be configured to determine a mean value for a tensive state classification range and determine the tensive state classification based on the mean value. In another or the same implementation, the control system is further configured to narrow a tensive state classification range.

Other innovative aspects of the subject matter described in this disclosure can be implemented in a method of using a photoacoustic signal to classify a tensive state of a user, the method comprising detecting an acoustic wave corresponding to a photoacoustic response of a blood vessel to light emitted by a light source system. The method may further include determining a wave characteristic from the acoustic wave and estimating a tensive state classification based on the determined wave characteristic. The tensive state classification is one of a plurality of tensive state classifications that each include different ranges of diastolic and systolic values.

According to a particular implementation, the method comprises determining a blood pressure reading based on the tensive state classification. Another example may include, based on the tensive state classification, estimating at least one of: demographic data, activity data, and real-time health data.

In another illustrative implementation, the method may include performing modelling operations on the acoustic wave to estimate a blood pressure reading. Other or the same implementations of the method may include selecting a predictive modelling operation from a plurality of predictive modelling operations based on at least one of the tensive state classification and the acoustic wave. An example may include mathematically weighting the wave characteristic to select a modelling operation from among a plurality of predictive modelling operations. Another or the same implementation may include accessing aggregated data from a database, where the aggregated data includes data from a plurality of users and at least one of their associated tensive state classifications and wave characteristics.

Implementations of the method may also include determining a mean value for a tensive state classification range and determining the tensive state classification based on the mean value. A tensive state classification range may be narrowed. In another or the same implementation, the method may include narrowing the tensive state classification range using the mean value.

Other innovative aspects of the subject matter described in this disclosure can be implemented in an apparatus that includes a means for detecting an acoustic wave corresponding to a photoacoustic response of a blood vessel to light emitted by a light source system, and a means for determining a wave characteristic from the acoustic wave and estimating a tensive state classification based on the determined wave characteristic, where the tensive state classification is a plurality of tensive state classifications that each include different ranges of diastolic and systolic values.

In some implementations, the means for determining the wave characteristic is further configured to determine a blood pressure reading based on the tensive state classification. The means for determining the wave characteristic may further be configured, based on the tensive state classification, to estimate at least one of: demographic data, activity data, and real-time health data.

In the same or other implementations, the means for determining the wave characteristic may be further configured to perform modelling operations on the acoustic wave to estimate a blood pressure reading. The means for determining the wave characteristic is further configured to select a predictive modelling operation from a plurality of predictive modelling operations based on at least one of the tensive state classification and the acoustic wave.

According to another particular aspect, the means for determining the wave characteristic is further configured to mathematically weight the wave characteristic to select a modelling operation from among a plurality of predictive modelling operations. The means for determining the wave characteristic may further be configured to access aggregated data from a database. The aggregated data includes data from a plurality of users and at least one of their associated tensive state classifications and wave characteristics. The means for determining the wave characteristic may further be configured to determine a mean value for a tensive state classification range and determine the tensive state classification based on the mean value.

Other innovative aspects of the subject matter described in this disclosure can be implemented in a computer-readable medium storing computer executable code for classifying a tensive state of a user, the computer executable code being configured to detect an acoustic wave corresponding to a photoacoustic response of a blood vessel to light emitted by a light source system, and determine a wave characteristic from the acoustic wave and estimate a tensive state classification based on the determined wave characteristic. The tensive state classification may be one of a plurality of tensive state classifications that each include different ranges of diastolic and systolic values.

According to a particular implementation, the computer-readable medium is further configured to, based on the tensive state classification, estimate at least one of: demographic data, activity data, and real-time health data. The computer-readable medium of an implementation may perform modelling operations on the acoustic wave to estimate a blood pressure reading.

Some or all of the methods described herein may be performed by one or more devices according to instructions (e.g., software) stored on non-transitory media. Such non-transitory media may include memory devices such as those described herein, including but not limited to random access memory (RAM) devices, read-only memory (ROM) devices, etc. Accordingly, some innovative aspects of the subject matter described in this disclosure can be implemented in one or more non-transitory media having software stored thereon. The software may include instructions for controlling one or more devices to perform one or more disclosed methods.

Details of one or more implementations of the subject matter described in this disclosure are set forth in the accompanying drawings and the description below. Other features, aspects, and advantages will become apparent from the description, the drawings, and the claims. Note that the relative dimensions of the following figures may not be drawn to scale.

### BRIEF DESCRIPTION OF THE DRAWINGS

FIG. 1 is a block diagram with illustrative components of an apparatus according to some disclosed implementations.

FIG. 2 is a block diagram of an illustrative system that includes a photoacoustic sensor and a local control system configured to output a blood pressure estimate based on a tensive state classification derived from a photoacoustic (e.g., PAPG) signal from the photoacoustic sensor.

FIG. 3 is a block diagram of an illustrative system that includes a photoacoustic device and a control system, in addition to a wireless connection to remote Internet servers and databases for the purpose of outputting a blood pressure estimate, among other estimated information, based on a tensive state classification.

FIG. 4 is a block diagram of an illustrative system that includes a photoacoustic device and a control system configured to determine the blood pressure and other estimates without using regression or other predictor models.

FIG. 5 is a block diagram of an apparatus configured to determine various estimations from classifying a tensive state based on an acoustic wave.

FIG. 6 is a chart depicting different tensive state classifications.

FIG. 7 shows examples of heart rate waveform (HRW) features that may be extracted according to some implementations of the systems and method described herein.

FIG. 8 shows examples of devices that may be used in a system for estimating blood pressure based, at least in part, on pulse transit time (PTT).

FIG. 9 shows a cross-sectional side view of a diagrammatic representation of a portion of an artery through which a pulse is propagating.

FIG. 10A shows an example ambulatory monitoring device designed to be worn around a wrist according to some implementations.

FIG. 10B shows an example ambulatory monitoring device designed to be worn on a finger according to some implementations.

FIG. 10C shows an example ambulatory monitoring device designed to reside on an earbud according to some implementations.

FIG. 11 is a flow diagram that shows examples of some disclosed operations.

FIG. 12 is a flow diagram that shows additional examples of some disclosed operations.

Like reference numbers and designations in the various drawings indicate like elements.

### DETAILED DESCRIPTION

The following description is directed to certain implementations for the purposes of describing various aspects of this disclosure. However, a person having ordinary skill in the art will readily recognize that the teachings herein can be applied in a multitude of different ways. Some of the concepts and examples provided in this disclosure are especially applicable to blood pressure monitoring applications. However, some implementations also may be applicable to other types of biological sensing applications, as well as to other fluid flow systems. The described implementations may be implemented in any device, apparatus, or system that includes an apparatus as disclosed herein. In addition, it is contemplated that the described implementations may be included in or associated with a variety of electronic devices such as, but not limited to: mobile telephones, multimedia Internet enabled cellular telephones, mobile television receivers, wireless devices, smartphones, smart cards, wearable devices such as bracelets, armbands, wristbands, rings, headbands, patches, etc., Bluetooth® devices, personal data assistants (PDAs), wireless electronic mail receivers, hand-held or portable computers, netbooks, notebooks, tablets, printers, copiers, scanners, facsimile devices, global positioning system (GPS) receivers/navigators, cameras, digital media players, game consoles, wrist watches, clocks, calculators, television monitors, flat panel displays, electronic reading devices (e.g., e-readers), mobile health devices, computer monitors, auto displays (including odometer and speedometer displays, etc.), cockpit controls and/or displays, camera view displays (such as the display of a rear view camera in a vehicle), architectural structures, microwaves, refrigerators, stereo systems, cassette recorders or players, DVD players, CD players, VCRs, radios, portable memory chips, washers, dryers, washer/dryers, parking meters, automobile doors, Internet of Things (IoT) devices, etc. Thus, the teachings are not intended to be limited to the specific implementations depicted and described with reference to the drawings; rather, the teachings have wide applicability as will be readily apparent to persons having ordinary skill in the art.

Tensive states are useful to classify blood pressure measurements into relatable, qualitative terminology that can be more meaningful to patients and medical professionals. Tensive states are conventionally determined by correlating blood pressure readings (i.e., diastolic and systolic pressure measurements) to a chart listing blood pressure ranges that account for both diastolic and systolic readings. The chart may include different ranges of diastolic and systolic values for each tensive state classification. The blood pressure measurements are generally acquired using a blood pressure cuff and a stethoscope. It is not always feasible or efficient to first measure blood pressures to determine tensive states classifications. For instance, it may not be practical to measure blood pressure while engaged in a hands-on activity, such as driving or working.

An implementation may determine a tensive state by applying classification processes to a received acoustic wave before and without having to first take a blood pressure readings. The acoustic wave may be used to classify the tensive state, rather than having to use a cuff or other measurement device to first measure blood pressure. More particularly, an implementation may use a photoacoustic plethysmogram (PAPG) signal to determine the tensive state rather than, and without having to acquire an actual blood pressure measurement (e.g., using a stethoscope and a blood pressure cuff). PAPG-capable devices have various potential advantages over more invasive health monitoring devices such as cuff-based or catheter-based blood pressure measurement devices. As used in an implementation, the tensive state classification generated by the tensive state classifier may function as a predictor of blood pressure and other health metrics. In other example, the tensive state classification may be used to estimate or predict user activities, as well as real-time health information and demographic data. Data may be exported to a third party for further analysis and use, such as a medical professional or navigational server. An estimated tensive state classification may additionally be used to select one or more computer models for use in determining blood pressure given inputs comprising measurements from the PAPG signal. In another example, the estimated tensive state classification may be used to determine the blood pressure given inputs comprising measurements from the PAPG signal without having to perform computer modelling. In either instance, an implementation may focus on identifying characteristics by mathematically weighting to more clearly distinguish the PAPG signal having the identifying characteristic from another PAPG signal having different characteristics.

Particular implementations of the subject matter described in this disclosure can be implemented to realize one or more of the following potential advantages. First classifying the tensive state according to the signal characteristics may improve the blood pressure estimation results. For instance, first classifying the tensive state may enable quicker blood pressure assessments and predictions for greater numbers of people. The tensive state classification may be used to make determinations regarding user activities, real-time health information, and non-health related information, such as demographic data used by third parties for marketing and service providing applications.

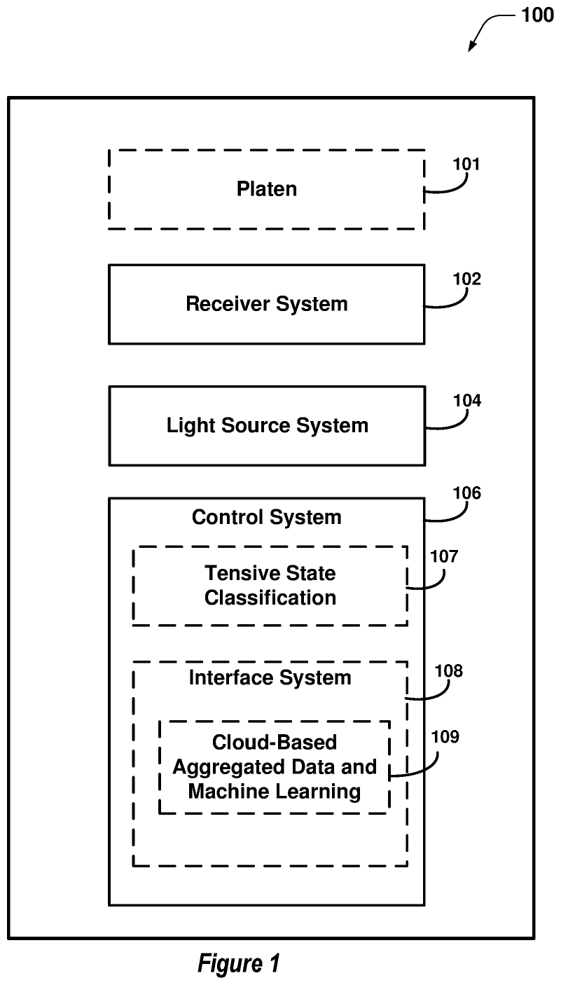

FIG. 1 is a block diagram that shows example components of an apparatus according to some disclosed implementations. As with the other components described with reference to all drawings described herein, the components of the apparatus represent modules that may be collocated or remote from the other modules in particular implementations. Turning more particularly to the example of FIG. 1, the apparatus 100 includes a platen 101, a receiver system 102, and a light source system 104. The particular implementation of the apparatus 100 may include a control system 106 comprising a tensive state classification module 107 and an interface system 108. Although the control system 106 is depicted by a single block in FIG. 1, the control system 106 of implementations may include one or more processors and associated electronics that work in combination, some being remote (connected via the interface system 108 to the Internet) and others that are local to a wearable photoacoustic device. Moreover, certain of the blocks of FIG. 1 are shown in dashed outline to denote they may be optional components.

The tensive state classification module 107 may be executed by the control system 106 to apply classification processes to a received acoustic wave before to classify the tensive state. The tensive state classification generated by the tensive state classification module 107 may function as a predictor of blood pressure, among other health and non-health related assessments.

The interface system 108 may include cloud-based aggregated data and machine learning 109. As described herein, the cloud-based implementation may allow data to be collected and stored from multiple users. The machine learning (e.g., deep learning) may train models using the aggregated data for each tensive state classification to learn trends and hone classification and estimations.

The platen 101 may be made of any suitable material, such as glass, acrylic, polycarbonate, etc. According to some examples, the platen 101 (or another portion of the apparatus) may include one or more anti-reflective layers. In some examples, one or more anti-reflective layers may reside on, or proximate, one or more outer surfaces of the platen 101.

In some examples, at least a portion of the outer surface of the platen 101 may have an acoustic impedance that is configured to approximate an acoustic impedance of human skin. The portion of the outer surface of the platen 101 may, for example, be a portion that is configured to receive a target object, such as a human digit. (As used herein, the terms “finger” and “digit” may be used interchangeably, such that a thumb is one example of a finger.) A typical range of acoustic impedances for human skin is 1.53-1.680 MRayls. In some examples, at least an outer surface of the platen 101 may have an acoustic impedance that is in the range of 1.4-1.8 MRayls, or in the range of 1.5-1.7 MRayls. Alternatively, or additionally, in some examples at least an outer surface of the platen 101 may be configured to conform to a surface of human skin. In some such examples, at least an outer surface of the platen 101 may have material properties like those of putty or chewing gum.

In some examples, at least a portion of the platen 101 may have an acoustic impedance that is configured to approximate an acoustic impedance of one or more receiver elements of the receiver system 102. According to some examples, a layer residing between the platen 101 and one or more receiver elements may have an acoustic impedance that is configured to approximate an acoustic impedance of the one or more receiver elements. Alternatively, or additionally, in some examples a layer residing between the platen 101 and one or more receiver elements may have an acoustic impedance that is in an acoustic impedance range between an acoustic impedance of the platen and an acoustic impedance of the one or more receiver elements. According to some examples, the receiver system 102 may include an array of receiver elements, in addition to receiver system circuitry.

In this implementation, the receiver system 102 is, or includes an ultrasonic receiver system. In some examples, the receiver system 102 may be configured to detect acoustic waves corresponding to a photoacoustic response of the target object to light emitted by the light source system. In some examples, the receiver system 102 may include a piezoelectric receiver layer, such as a layer of polyvinylidene difluoride (PVDF) polymer, polyvinylidene fluoride-trifluoroethylene (PVDF-TrFE) copolymer, a piezoelectric composite, etc. In some implementations, a single piezoelectric layer may serve as an ultrasonic receiver. In some implementations, other piezoelectric materials may be used in the piezoelectric layer, such as aluminum nitride (AlN) or lead zirconate titanate (PZT). The receiver system 102 may, in some examples, include an array of ultrasonic transducer elements, such as an array of piezoelectric micromachined ultrasonic transducers (PMUTs), an array of capacitive micromachined ultrasonic transducers (CMUTs), etc. In some such examples, a piezoelectric receiver layer, PMUT elements in a single-layer array of PMUTs, or CMUT elements in a single-layer array of CMUTs, may be used as ultrasonic transmitters as well as ultrasonic receivers. According to some examples, the receiver system 102 may be, or may include an ultrasonic receiver array. In some examples, the apparatus 100 may include one or more separate ultrasonic transmitter elements.

In some such examples, the ultrasonic transmitter(s) may include an ultrasonic plane-wave generator. In some examples, the receiver system 102 may include an optical receiver system. According to some examples, the light source system 104 may include a light-emitting component and light source system circuitry . . . .

The light source system 104 may, in some examples, include one or more light-emitting diodes. In some implementations, the light source system 104 may include one or more laser diodes. According to some implementations, the light source system 104 may include one or more vertical-cavity surface-emitting lasers (VCSELs). In some implementations, the light source system 104 may include one or more edge-emitting lasers. In some implementations, the light source system may include one or more neodymium-doped yttrium aluminum garnet (Nd:YAG) lasers.

The light source system 104 may, in some examples, be configured to transmit light in one or more wavelength ranges. In some examples, the light source system 104 may configured for transmitting light in a wavelength range of 500 to 600 nanometers. According to some examples, the light source system 104 may configured for transmitting light in a wavelength range of 800 to 950 nanometers.

The light source system 104 may include various types of drive circuitry, depending on the particular implementation. In some disclosed implementations, the light source system 104 may include at least one multi-junction laser diode, which may produce less noise than single-junction laser diodes. In some examples, the light source system 104 may include a drive circuit (also referred to herein as drive circuitry) configured to cause the light source system to emit pulses of light at pulse widths in a range from 3 nanoseconds to 1000 nanoseconds. According to some examples, the light source system 104 may include a drive circuit configured to cause the light source system to emit pulses of light at pulse repetition frequencies in a range from 1 kilohertz to 100 kilohertz.

In some implementations, the light source system 104 may be configured for emitting various wavelengths of light, which may be selectable to trigger acoustic wave emissions primarily from a particular type of material. For example, because the hemoglobin in blood absorbs near-infrared light very strongly, in some implementations the light source system 104 may be configured for emitting one or more wavelengths of light in the near-infrared range, in order to trigger acoustic wave emissions from hemoglobin. However, in some examples the control system 106 may control the wavelength(s) of light emitted by the light source system 104 to preferentially induce acoustic waves in blood vessels, other soft tissue, and/or bones. For example, an infrared (IR) light-emitting diode LED may be selected, and a short pulse of IR light emitted to illuminate a portion of a target object and generate acoustic wave emissions that are then detected by the receiver system 102. In another example, an IR LED and a red LED or other color such as green, blue, white, or ultraviolet (UV) may be selected and a short pulse of light emitted from each light source in turn with ultrasonic images obtained after light has been emitted from each light source. In other implementations, one or more light sources of different wavelengths may be fired in turn or simultaneously to generate acoustic emissions that may be detected by the ultrasonic receiver. Image data from the ultrasonic receiver that is obtained with light sources of different wavelengths and at different depths (e.g., varying RGDs) into the target object may be combined to determine the location and type of material in the target object. Image contrast may occur as materials in the body generally absorb light at different wavelengths differently. As materials in the body absorb light at a specific wavelength, they may heat differentially and generate acoustic wave emissions with sufficiently short pulses of light having sufficient intensities. Depth contrast may be obtained with light of different wavelengths and/or intensities at each selected wavelength. That is, successive images may be obtained at a fixed RGD (which may correspond with a fixed depth into the target object) with varying light intensities and wavelengths to detect materials and their locations within a target object. For example, hemoglobin, blood glucose or blood oxygen within a blood vessel inside a target object such as a finger may be detected photoacoustically.

According to some implementations, the light source system 104 may be configured for emitting a light pulse with a pulse width less than about 100 nanoseconds. In some implementations, the light pulse may have a pulse width between about 10 nanoseconds and about 500 nanoseconds or more. According to some examples, the light source system may be configured for emitting a plurality of light pulses at a pulse repetition frequency between 10 Hz and 100 kHz. Alternatively, or additionally, in some implementations the light source system 104 may be configured for emitting a plurality of light pulses at a pulse repetition frequency between about 1 MHz and about 100 MHz. Alternatively, or additionally, in some implementations the light source system 104 may be configured for emitting a plurality of light pulses at a pulse repetition frequency between about 10 Hz and about 1 MHz. In some examples, the pulse repetition frequency of the light pulses may correspond to an acoustic resonant frequency of the ultrasonic receiver and the substrate. For example, a set of four or more light pulses may be emitted from the light source system 104 at a frequency that corresponds with the resonant frequency of a resonant acoustic cavity in the sensor stack, allowing a build-up of the received ultrasonic waves and a higher resultant signal strength. In some implementations, filtered light or light sources with specific wavelengths for detecting selected materials may be included with the light source system 104. In some implementations, the light source system may contain light sources such as red, green, and blue LEDs of a display that may be augmented with light sources of other wavelengths (such as IR and/or UV) and with light sources of higher optical power. For example, high-power laser diodes or electronic flash units (e.g., an LED or xenon flash unit) with or without filters may be used for short-term illumination of the target object.

In some implementations, the apparatus (for example, the receiver system 102, the light source system 104, or both) may include one or more sound-absorbing layers, acoustic isolation material, light-absorbing material, light-reflecting material, or combinations thereof. In some examples, acoustic isolation material may reside between the light source system 104 and at least a portion of the receiver system 102. In some examples, the apparatus (for example, the receiver system 102, the light source system 104, or both) may include one or more electromagnetically shielded transmission wires. In some such examples, the one or more electromagnetically shielded transmission wires may be configured to reduce electromagnetic interference from the light source system 104 that is received by the receiver system 102.

The control system 106 may include one or more general purpose single- or multi-chip processors, digital signal processors (DSPs), application specific integrated circuits (ASICs), field programmable gate arrays (FPGAs) or other programmable logic devices, discrete gates or transistor logic, discrete hardware components, or combinations thereof. The control system 106 also may include (and/or be configured for communication with) one or more memory devices, such as one or more random access memory (RAM) devices, read-only memory (ROM) devices, etc. Accordingly, the apparatus 100 may have a memory system that includes one or more memory devices, though the memory system is not shown in FIG. 1. The control system 106 may be configured for receiving and processing data from the receiver system 102, e.g., as described below. If the apparatus 100 includes an ultrasonic transmitter, the control system 106 may be configured for controlling the ultrasonic transmitter. In some implementations, functionality of the control system 106 may be partitioned between one or more controllers or processors, such as a dedicated sensor controller and an applications processor of a mobile device.

In some examples, the control system 106 may be configured to control the light source system 104. For example, the control system 106 may be configured to control one or more light-emitting portions of the light source system 104 to emit laser pulses. The laser pulses may, in some examples, be in a wavelength range of 600 nm to 1000 nm. The laser pulses may, in some examples, have pulse widths in a range from 3 nanoseconds to 1000 nanoseconds. In some examples, the control system 106 may be configured to receive signals from the ultrasonic receiver system 102 corresponding to the ultrasonic waves generated by the target object responsive to the light from the light source system 104. In some examples, the control system 106 may be configured to estimate one or more cardiac features based, at least in part, on the signals. According to some examples, the cardiac features may be, or may include blood pressure.

Some implementations of the apparatus 100 may include the interface system 108. In some examples, the interface system 108 may include a wireless interface system. In some implementations, the interface system 108 may include a user interface system, one or more network interfaces, one or more interfaces between the control system 106 and a memory system and/or one or more interfaces between the control system 106 and one or more external device interfaces (e.g., ports or applications processors), or combinations thereof. According to some examples in which the interface system 108 is present and includes a user interface system, the user interface system may include a microphone system, a loudspeaker system, a haptic feedback system, a voice command system, one or more displays, or combinations thereof. According to some examples, the interface system 108 may include a touch sensor system, a gesture sensor system, or a combination thereof. The touch sensor system (if present) may be, or may include, a resistive touch sensor system, a surface capacitive touch sensor system, a projected capacitive touch sensor system, a surface acoustic wave touch sensor system, an infrared touch sensor system, any other suitable type of touch sensor system, or combinations thereof.

In some examples, the interface system 108 may include a force sensor system. The force sensor system (if present) may be, or may include, a piezo-resistive sensor, a capacitive sensor, a thin film sensor (for example, a polymer-based thin film sensor), another type of suitable force sensor, or combinations thereof. If the force sensor system includes a piezo-resistive sensor, the piezo-resistive sensor may include silicon, metal, polysilicon, glass, or combinations thereof. In some examples, the interface system 108 may include an optical sensor system, one or more cameras, or a combination thereof.

The apparatus 100 may be used in a variety of different contexts, many examples of which are disclosed herein. For example, in some implementations a mobile device may include the apparatus 100. In some such examples, the mobile device may be a smart phone. In some implementations, a wearable device may include the apparatus 100. The wearable device may, for example, be a bracelet, an armband, a wristband, a watch, a ring, a headband, or a patch.

Several implementation are described herein that illustrate various stack configurations of receiver systems. Each receiver system described herein includes an array of receiver elements, in addition to receiver system circuitry. Not all of the components of a PAPG sensors are shown in each illustration, as the drawings are intended to include those components useful in describing the context and functionality of the features. Further, the respective thicknesses and other dimensions of the different stacked layers described herein are not to scale.

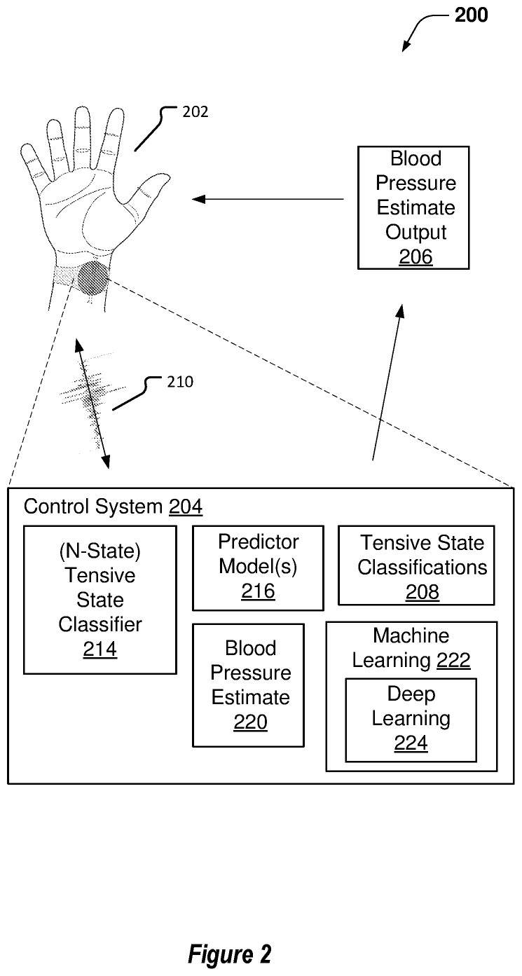

FIG. 2 is a block diagram of an illustrative apparatus 200 that includes a photoacoustic device 202 and a control system 204 configured to output a blood pressure estimate 206 based on a tensive state classification 208. The tensive state classification 208 may be derived from a photoacoustic (e.g., PAPG) signal 210 from a sensor of the photoacoustic device 202. In other implementations described hereafter, a control system may include one or more processors and associated electronics that are remote from the photoacoustic device (e.g., connected via the Internet). In the implementation depicted in FIG. 2, the control system 204 may be included within or be proximate the photoacoustic device 202, as signified by the dashed lines. According to an implementation, the control system 204 is an instance of control system 106 of FIG. 1.

The control system 204 may include a (multi-state) tensive state classifier 214, and one or more predictor models 216. The control system 204 may be configured to detect a signal 210 from a receiver system corresponding to an acoustic wave. The acoustic wave may correspond to a photoacoustic response of a blood vessel to light emitted by a light source system of the photoacoustic device 202. Described in greater detail in subsequent figures, the light source system may include a light-emitting component.

More particularly, the tensive state classifier 214 may determine a wave characteristic from the acoustic wave signal 210 and estimate a tensive state classification 208 based on the determined wave characteristic. The tensive state classification 208 may be one of a plurality of tensive state classifications that each include different ranges of diastolic and systolic values. In one example, an implementation may use three illustrative tensive state classifications: high, medium, and low. Each of the tensive state classifications may include both systolic and diastolic inputs. Other implementations may include fewer or more tensive state classifications as useful.

The tensive state classifier 214 of an implementation may be configured to select a modelling operation from a plurality of modelling operations (i.e., predictor models 216) based on at least one of the tensive state classification 208 and the acoustic wave signal 210. The selected predictor model(s) 216 may perform modelling operations on the pattern and or other characteristics of the acoustic wave signal 210 to estimate the blood pressure reading 220.

Certain predictor models 216 may be more effective when executing data associated in different types of photoacoustic signal patterns. The tensive state classifier 214 may include artificial intelligence, such as machine learning and deep learning algorithms 222, 224, to predict the tensive state classification 208 based on an evaluation of the photoacoustic signal 210 (e.g., signal pattern and/or signal characteristics). The tensive state classifier 214 may use feedback from the artificial intelligence (and learned processes) over time to select a most effective blood pressure predictor model 216.

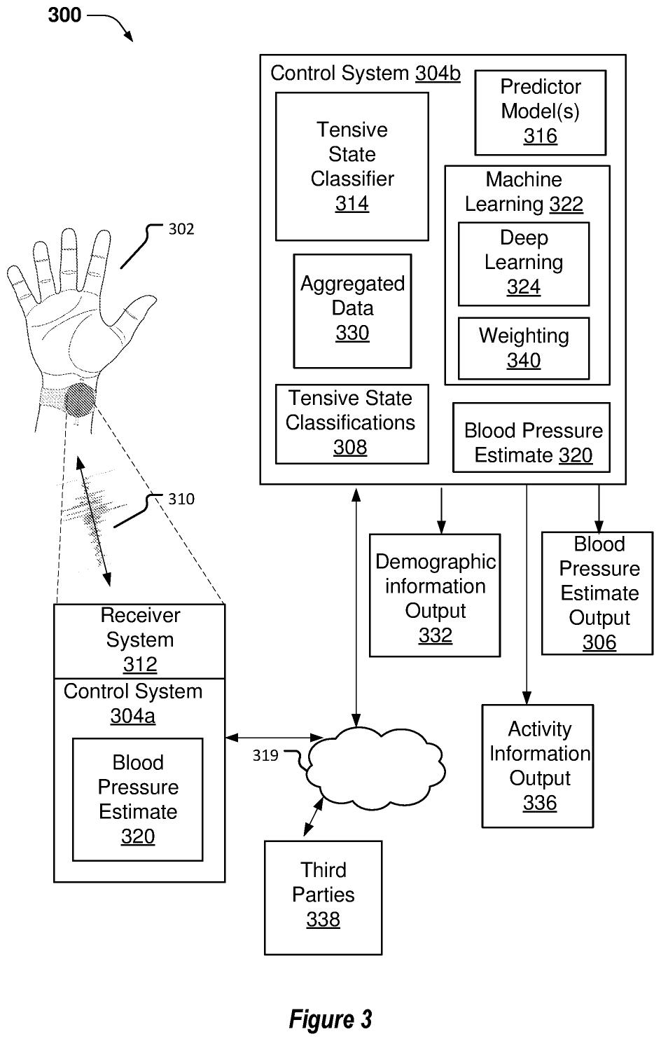

FIG. 3 is a block diagram of an illustrative system 300 that includes a photoacoustic device 302 and a control system 304a and 304b, in addition to a wireless connection to remote Internet servers and databases. The system 300 may output a blood pressure estimate 306, among other estimated information, based on a tensive state classification 308.

While tensive state classification processes may be performed on a worn device, the implementation of FIG. 3 enables part or all of the classification to be performed at a server in communication with the worn device. A cloud-based implementation may allow data to be aggregated data 330 from multiple users to learn more trends. For example, deep learning processes 324 may determine if the blood pressure increases for certain populations at certain locations or during specific times of day due to external factors like traffic jams and roadblocks. An implementation may tie in with other applications (e.g., a navigational app) to suggest different routes or times to perform an activity based on the predicted tensive classification state of a user and others being monitored via their PAPG signals.

A tensive state classifier 314 may process different signal characteristics (e.g., peaks, valleys, periodicity, wavelength, amplitude, slope, among other traits) that can be logically associated with a range of the estimated tensive state classifications 308, and ultimately, to real-time blood pressure estimations 320. The estimated tensive state classifications affect or otherwise relate to blood pressure. The tensive state estimations based on PAPG signal evaluation may additionally enable activity tracking estimations.

More particularly, the predicted tensive state classifications 308 may be used to estimate activity information outputs 336. In one example, the system may receive real-time PAPG signals from a group of people wearing photoacoustic sensors. The system 300 may be able to assess from the subsequently estimated tensive classification states that the members of the group are commuting. For instance, the estimated tensive state classification 308 may show an increasing trend (e.g., perhaps associated with driving in traffic). Continuing with the example, the activity assessment (e.g., commuting) may be combined with other data (e.g., geographic locational data) to recommend alternative routes for commuters via a navigational application interface.

In another example, a signal may have characteristics particular to a post-meal PAPG signal pattern that is associated with a medium tensive state estimation. A premeal PAPG signal pattern may reveal a likely low tensive state. As described herein, certain signal characteristics of a PAPG signal may correspond to a user driving. A spike in another PAPG signal may reveal that a user is exercising on a treadmill.

In another application, a predicted elevated, tensive state may be used to alert a user or medical professional of a potential, imminent medical emergency, such as a heart attack, stroke, or drug use. For instance, the PAPG signal tensive state classifications may be used to detect and track heart rate variability, cardiac output, drug ingestion, and heart conditions (e.g., arrhythmia).

The estimated tensive classification 308 may also be used to predict other non-health related metrics, such as demographic information output 332. For instance, the system 300 may predict that a person having a tensive classification or PAPG signal pattern parameter indicative of travel along a known route at a known time may be a school age child. For example, the detected travel may coincide with the timing of known bus routes in the afternoon. Such medical and non-medical data may be exported for further analysis and use to a third party 338, such as a medical professional or navigational server.

As described herein, the tensive state classifications 308 may be derived from the photoacoustic (e.g., PAPG) signal 310 from a sensor (not shown) of the photoacoustic device 302. The PAPG signal 310 may more particularly be acoustic waves resulting from a photoacoustic response of the target object that are received by the receiver system 312. In other words, the receiver system 312 may be the sensor that receives the acoustic/PAPG signal 310. The control system 304a, 304b is depicted in different blocks to illustrate that one or more processors and associated electronics may work in combination, some being remote (e.g., 304b connected via the Internet) and others that are local (e.g., 304a) to the photoacoustic device 302. According to an implementation, the control system 304a, 304b is an instance of control system 106 of FIG. 1.

The control system 304a may locally stored blood pressure estimates 320 downloaded from the Internet. A receiver system 312 may be configured to detect an acoustic wave comprising the signal 310. The acoustic wave may correspond to a photoacoustic response of a blood vessel to light emitted by a light source system of the photoacoustic device 302.

The remotely situated control system 304b may include a (multi-state) tensive state classifier 314, and one or more predictor models 316. The tensive state classifier 314 may determine a wave characteristic from the uploaded acoustic wave signal 310 and estimate a tensive state classification 308 based on the determined wave characteristic. The tensive state classification 308 may be one of a plurality of tensive state classifications that each include different ranges of diastolic and systolic values.

The tensive state classifier 314 of an implementation may be configured to select a modelling operation from a plurality of modelling operations (i.e., the predictor models 316) based on at least one of the tensive state classification 308 and the acoustic wave signal 310. The selected predictor model(s) 316 may perform modelling operations on the pattern and or other characteristics of the acoustic wave signal 310 to estimate the blood pressure reading 320.

Certain predictor models 316 may be more effective when executing data associated in different types of photoacoustic signal patterns. The tensive state classifier 314 may include artificial intelligence, such as machine learning and deep learning algorithms 322, 324, to predict the tensive state classification 308 based on an evaluation of the photoacoustic signal 310 (e.g., signal pattern and/or signal characteristics). The tensive state classifier 314 may use feedback from the artificial intelligence and learned processes using the aggregated data 330 over time to select a most effective blood pressure predictor model 316.

An implementation may further classify the signal by using a weighted scheme 340. For example, an implementation of a particular blood pressure predictor 316 may include weighted deep learning processes 324 that are each specific to one of the multiple tensive state classifications 308 to estimate a brachial blood pressure. The system 300 may train the machine learning/deep learning models 324 for each tensive state classification 308. Different blood pressure predictor models 316 may use different feature sets, or signal characteristics. Different blood pressure predictors 316 may use the different weighting scheme 340 for common features (e.g., learned to be associated with tensive state classifications 308).

The computer models may be logically linked or otherwise associated in storage to the different predictor models 316. As such, the predictor models 316 are likewise also linked to the associated signal characteristics. As described in the following figure, other implementations may determine the blood pressure and other estimates without using a predictor model 316 by logically linking or otherwise associating wave signal characteristics signal characteristics (e.g., peaks, valleys, periodicity, wavelength, amplitude, slope, among other traits) with the tensive state classifications 308.

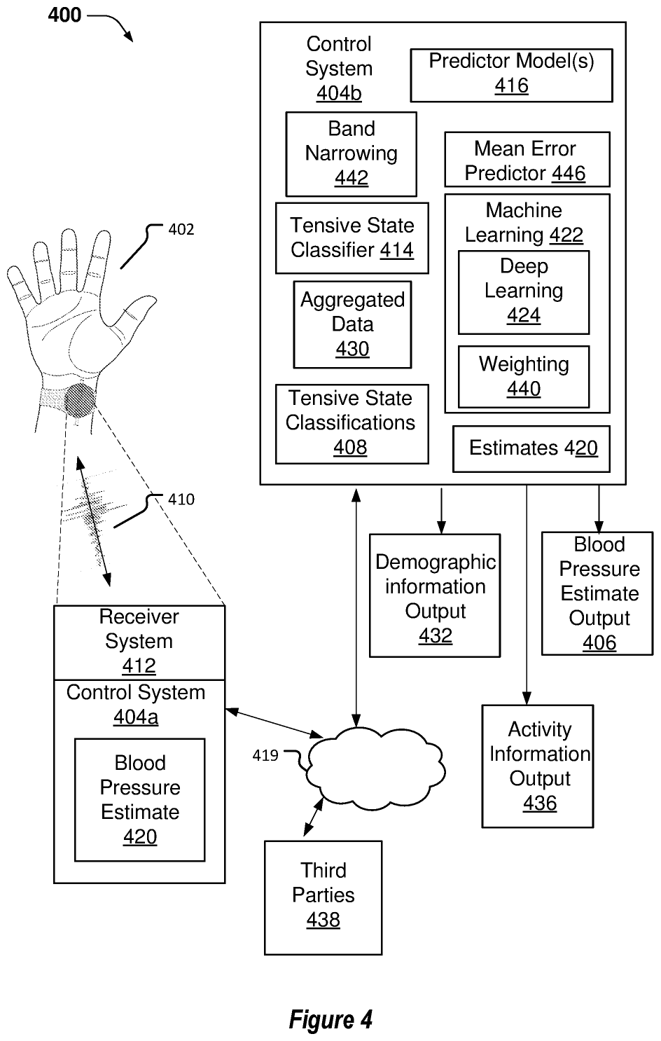

FIG. 4 is a block diagram of an illustrative system 400 that includes a photoacoustic device 402 and a control system 404a, 404b configured to determine the blood pressure and other estimates without using regression or other predictor models. In the implementation of FIG. 4, the system 400 additionally includes a wireless connection to remote Internet servers and databases. The system 400 may output a blood pressure estimate 406, among other estimated information, based on a tensive state classification 408.

An implementation may predict, or estimate, blood pressure data using only the tensive state classifier 414 (e.g., without a regression model). In such a scenario, the tensive state band may be dynamically adjusted by a band narrowing algorithm 442 to be narrow enough to use a mean error predictor 446 for the blood pressure estimate 406. That is, if the range of the tensive state band is sufficiently narrow, the estimation may have greater veracity.

More specifically, the class mean predictor 446 may be used to determine a mean value for a tensive classification range. The mean value may be used as a rough prediction. The rough prediction may then be refined based on certain feature values measured from the PAPG signal. For example, an arterial diameter measurement might be used to refine the prediction. In this manner, the blood pressure estimator 406 may be determined in a manner that does not use machine learning/deep learning predictors.

The configuration of FIG. 4 enables part or all of the tensive state classification processes to be performed at a server in communication with the worn device. However, another implementation may be performed locally on the worn device. The cloud-based implementation may allow data to be aggregated data 430 from multiple users to learn more trends. An implementation may tie in with other third-party entities 438 to garner different estimations as described herein.

The tensive state classifier 414 may process different signal characteristics (e.g., peaks, valleys, periodicity, wavelength, amplitude, slope, among other traits) that can be logically associated with a range of the estimated tensive state classifications 408, and ultimately, to real-time blood pressure estimations 420. The estimated tensive state classifications affect or otherwise relate to blood pressure. The tensive state estimations based on PAPG signal evaluation may additionally enable activity tracking estimations as described herein.

The tensive state classifications 408 may be derived from the photoacoustic (e.g., PAPG) signal 410 from a sensor (not shown) of the photoacoustic device 402. The control system 404a, 404b is depicted in different blocks to illustrate that one or more processors and associated electronics may work in combination, some being remote (e.g., 404b connected via the Internet) and others that are local (e.g., 404b) to the photoacoustic device 402. According to an implementation, the control system 404a, 404b is an instance of control system 106 of FIG. 1.

The control system 404a may include locally stored blood pressure estimates 420 downloaded from the Internet. A receiver system 412 may be configured to detect an acoustic wave comprising the signal 410. The acoustic wave may correspond to a photoacoustic response of a blood vessel to light emitted by a light source system of the photoacoustic device 402.

The remotely situated control system 404b may include a (multi-state) tensive state classifier 414. The tensive state classifier 414 may determine a wave characteristic from the uploaded acoustic wave signal 410 and estimate a tensive state classification 408 based on the determined wave characteristic. The tensive state classification 408 may be one of a plurality of tensive state classifications that each include different ranges of diastolic and systolic values.

The tensive state classifier 414 of an implementation may be configured to select a tensive state classification 408 based on the acoustic wave signal 410. The selected tensive state classification 408 (determined based on the pattern and or other characteristics of the acoustic wave signal 410) may estimate the blood pressure reading 420. The signal traits/characteristics (peaks, valleys, periodicity, wavelength, amplitude, slope) may be logically associated with a range of estimated tensive state classifications 408, and ultimately, to blood pressure estimation outputs 406. The classifications may further be associated with potential environmental triggers and other data that affect or otherwise relate to blood pressure.

The tensive state classifier 414 may include artificial intelligence, such as machine learning and deep learning algorithms 422, 424, to predict the tensive state classification 408 based on an evaluation of the acoustic wave signal 410 (e.g., signal pattern and/or signal characteristics). The tensive state classifier 414 may use feedback from the artificial intelligence and learned processes using the aggregated data 430 over time to select a most effective blood pressure predictor model 416.

An implementation may further classify the signal by using a weighted scheme, or algorithm 440. For example, an implementation of a particular tensive state classifier 414 may include weighted deep learning processes 424 that are each specific to one of the multiple tensive state classifications 408 to estimate a brachial blood pressure. The system 400 may train the tensive state classifier 414 for each tensive state classification 408. Different weighting schemes and algorithms 440 may be used for common features learned to be associated with tensive state classifications 408. Features, or characteristics, of a PAPG signal may thus be segregated and potentially weighted based on established tensive classes. Some characteristics of the PAPG signal may be more indicative than others with regard identifying tensive classes.

An implementation may focus on that characteristic (e.g., by mathematical weighting) to distinguish the PAPG signal having the identifying characteristic more clearly from another PAPG signal having a different characteristic. For example, PAPG signal data may be represented in an equation with individual values corresponding to the identifying characteristics being multiplied or otherwise increased. The weighting and distinguishing may enable the PAPG signal to be correctly correlated to the appropriate tensive state.

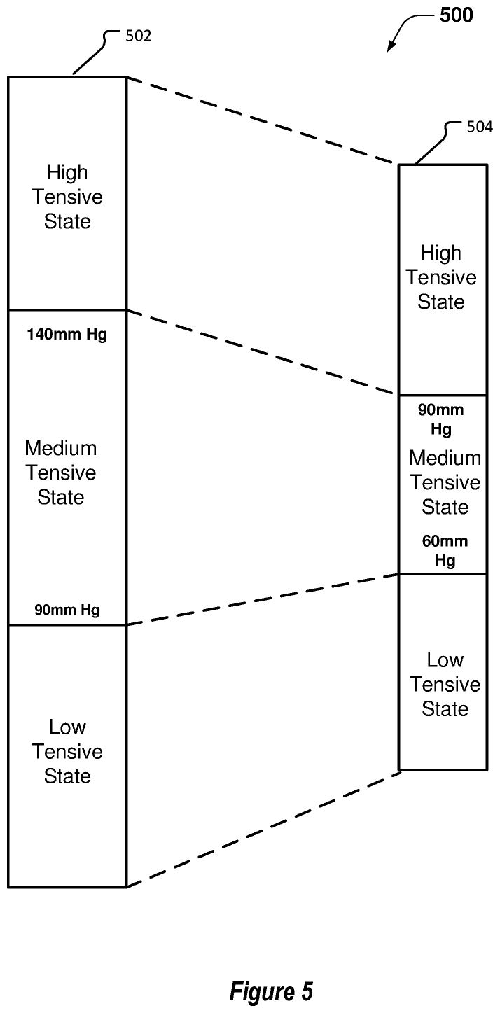

FIG. 5 is a chart 500 showing illustrative tensive state classifications. An implementation may include more or fewer tensive state classifications. For illustrative purposes, the implementation of FIG. 5 uses only three tensive state classifications: high, medium, and low. Other implementations may include fewer or more tensive state classifications as useful. Each of the tensive state classifications may include both systolic 502 and diastolic data 504.

In the example of FIG. 5, blood pressure readings that include systolic pressure measurements between 140 hectograms (Hg) and 90 Hg may be classified as a medium tensive state. Systolic pressure measurements above 140 Hg may be classified as high, and below 90 Hg may be classified as a low tensive state. Blood pressure readings that include diastolic pressure measurements between 90 hectograms (Hg) and 60 Hg may be classified as a medium tensive state. Systolic pressure measurements above 90 Hg may be classified as high, and below 60 Hg may be classified as a low tensive state.

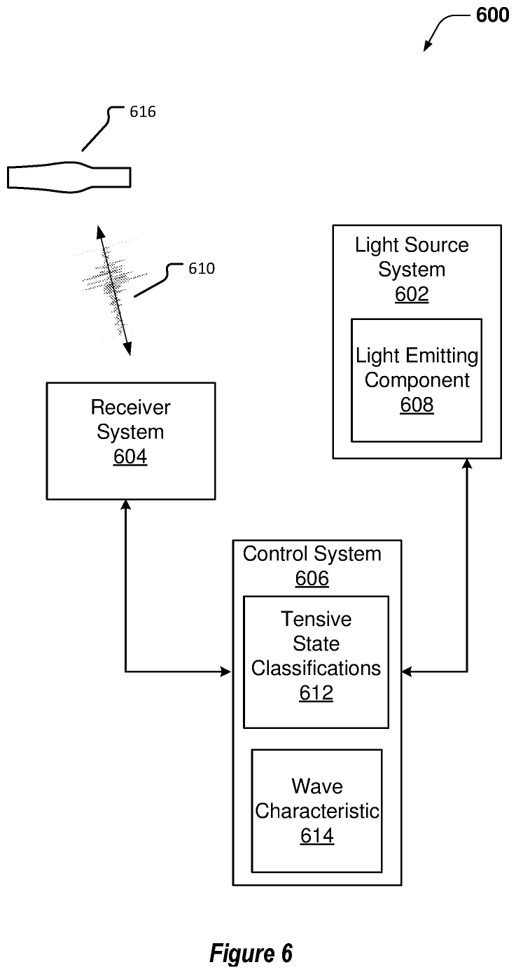

FIG. 6 is a block diagram of an apparatus 600 that includes a light source system 602, a receiver system 604, and a control system 606. The light source system 602 includes a light-emitting component 608. The receiver system 604 may be configured to detect an acoustic wave 610 corresponding to a photoacoustic response of a blood vessel to light emitted by the light source system 602. The control system 606 may be configured to determine a wave characteristic 614 from the acoustic wave 610 and to estimate a tensive state classification 612 based on the determined wave characteristic 614. The control system 606 may be an instance of the control system 106 of FIG. 1. The tensive state classification 612 may be one of a plurality of tensive state classifications 612 that each include different ranges of diastolic and systolic values.

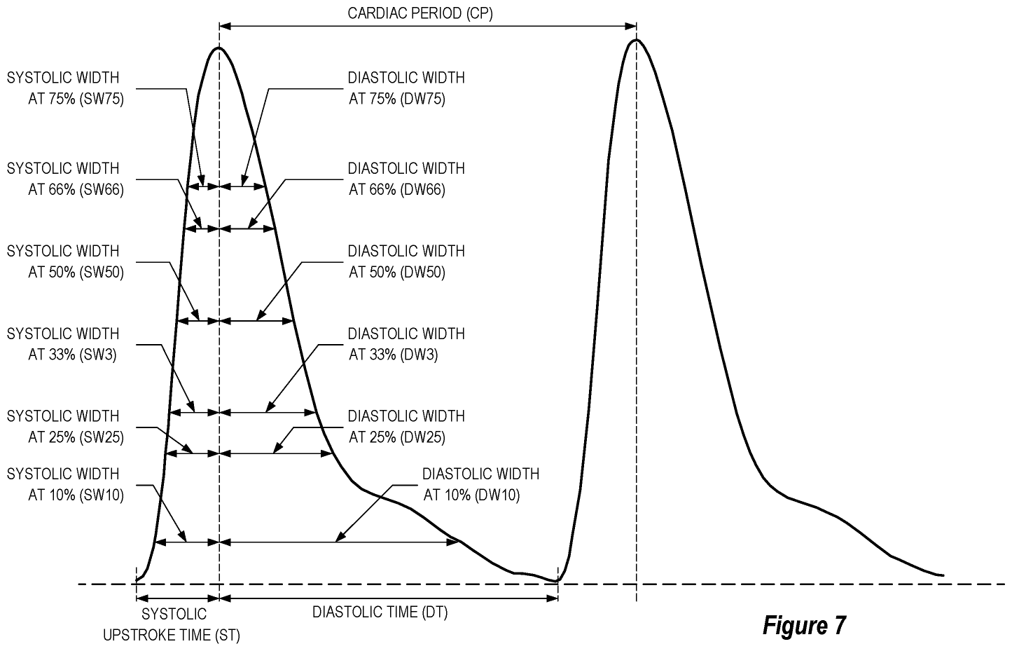

FIG. 7 shows examples of heart rate waveform (HRW) features that may be extracted according to some implementations of the systems and method described herein. The horizontal axis of FIG. 7 represents time, and the vertical axis represents signal amplitude. The cardiac period is indicated by the time between adjacent peaks of the HRW. The systolic and diastolic time intervals are indicated below the horizontal axis. During the systolic phase of the cardiac cycle, as a pulse propagates through a particular location along an artery, the arterial walls expand according to the pulse waveform and the elastic properties of the arterial walls. Along with the expansion is a corresponding increase in the volume of blood at the particular location or region, and with the increase in volume of blood an associated change in one or more characteristics in the region. Conversely, during the diastolic phase of the cardiac cycle, the blood pressure in the arteries decreases and the arterial walls contract. Along with the contraction is a corresponding decrease in the volume of blood at the particular location, and with the decrease in volume of blood an associated change in the one or more characteristics in the region.

The HRW features that are illustrated in FIG. 7 pertain to the width of the systolic and/or diastolic portions of the HRW curve at various “heights,” which are indicated by a percentage of the maximum amplitude. For example, the SW50 feature is the width of the systolic portion of the HRW curve at a “height” of 50% of the maximum amplitude. In some implementations, the HRW features used for blood pressure estimation may include some or all of the SW10, SW25, SW33, SW50, SW66, SW75, DW10, DW25, DW33, DW50, DW66 and DW75 HRW features. In other implementations, additional HRW features may be used for blood pressure estimation. Such additional HRW features may, in some instances, include the sum and ratio of the SW and DW at one or more “heights,” e.g., (DW75+SW75), DW75/SW75, (DW66+SW66), DW66/SW66, (DW50+SW50), DW50/SW50, (DW33+SW33), DW33/SW33, (DW25+SW25), DW25/SW25 and/or (DW10+SW10), DW10/SW10. Other implementations may use yet other HRW features for blood pressure estimation. Such additional HRW features may, in some instances, include sums, differences, ratios and/or other operations based on more than one “height,” such as (DW75+SW75)/(DW50+SW50), (DW50+SW50/(DW10+SW10), etc.

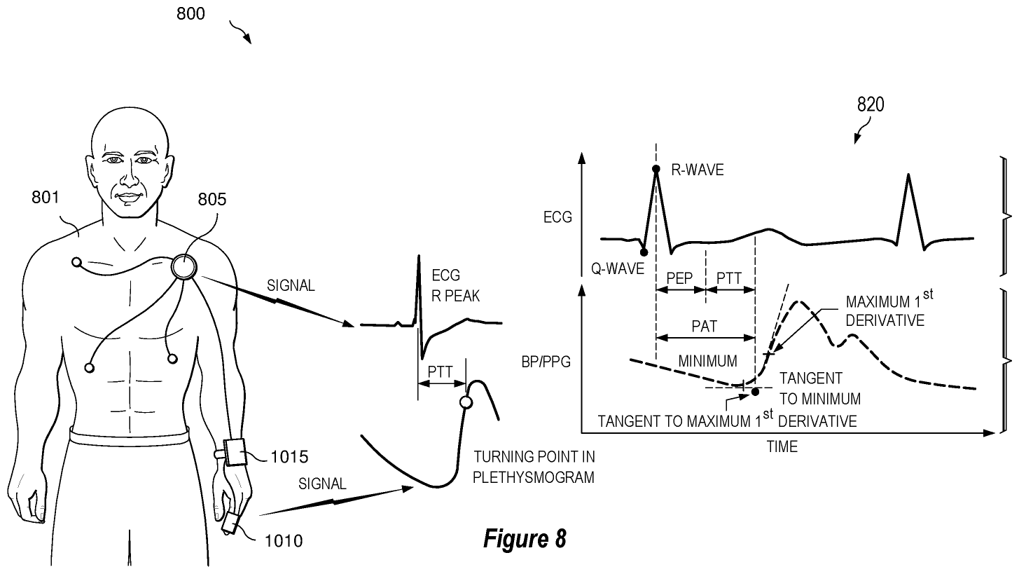

FIG. 8 shows examples of devices that may be used in a system for estimating blood pressure based, at least in part, on pulse transit time (PTT). As with other figures provided herein, the numbers, types and arrangements of elements are merely presented by way of example. According to this example, the system 800 includes at least two sensors. In this example, the system 800 includes at least an electrocardiogram sensor 805 and a device 810 that is configured to be mounted on a finger of the person 801. In this example, the device 810 is, or includes, an apparatus configured to perform at least some PAPG methods disclosed herein. For example, the device 810 may be, or may include the apparatus 80 of FIG. 1 or a similar apparatus.

As noted in the graph 820, the PAT includes two components, the pre-ejection period (PEP, the time needed to convert the electrical signal into a mechanical pumping force and isovolumetric contraction to open the aortic valves) and the PTT. The starting time for the PAT can be estimated based on the QRS complex—an electrical signal characteristic of the electrical stimulation of the heart ventricles. As shown by the graph 820, in this example the beginning of a pulse arrival time (PAT) may be calculated according to an R-Wave peak measured by the electrocardiogram sensor 805 and the end of the PAT may be detected via analysis of signals provided by the device 810. In this example, the end of the PAT is assumed to correspond with an intersection between a tangent to a local minimum value detected by the device 810 and a tangent to a maximum slope/first derivative of the sensor signals after the time of the minimum value.

There are many known algorithms for blood pressure estimation based on the PTT and/or the PAT, some of which are summarized in Table 1 and described in the corresponding text on pages 5-10 of Sharma, M., et al., Cuff-Less and Continuous Blood Pressure Monitoring: A Methodological Review (“Sharma”), in Multidisciplinary Digital Publishing Institute (MDPI) Technologies 2017, 5, 21, both of which are hereby incorporated by reference.

Some previously-disclosed methods have involved calculating blood pressure according to one or more of the equations shown in Table 1 of Sharma, or other known equations, based on a PTT and/or PAT measured by a sensor system that includes a PPG sensor. As noted above, some disclosed PAPG-based implementations are configured to distinguish artery HRWs from other HRWs. Such implementations may provide more accurate measurements of the PTT and/or PAT, relative to those measured by a PPG sensor. Therefore, disclosed PAPG-based implementations may provide more accurate blood pressure estimations, even when the blood pressure estimations are based on previously-known formulae.

Other implementations of the system 800 may not include the electrocardiogram sensor 805. In some such implementations, the device 815, which is configured to be mounted on a wrist of the person 801, may be, or may include, an apparatus configured to perform at least some PAPG methods disclosed herein. For example, the device 815 may be, or may include the apparatus 200 of FIG. 2 or a similar apparatus. According to some such examples, the device 815 may include a light source system and two or more ultrasonic receivers. One example is described below with reference to FIG. 15A. In some examples, the device 815 may include an array of ultrasonic receivers.

In some implementations of the system 800 that do not include the electrocardiogram sensor 805, the device 810 may include a light source system and two or more ultrasonic receivers. One example is described below with reference to FIG. 15B.

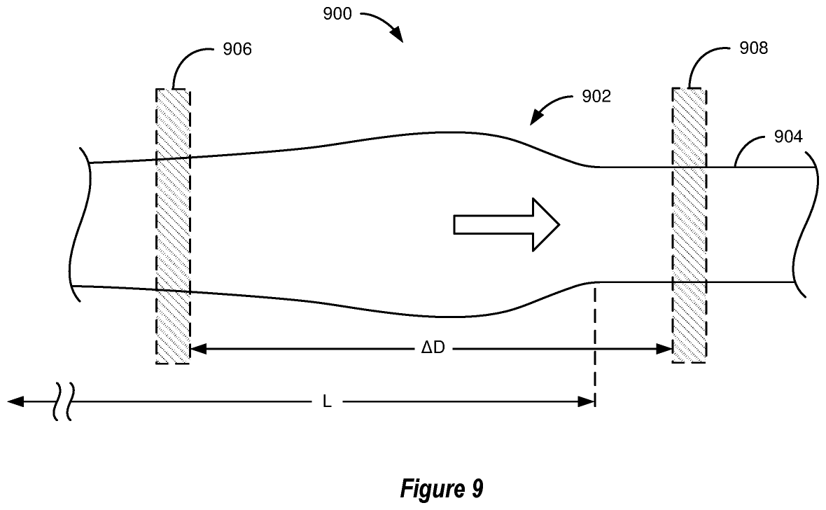

FIG. 9 shows a cross-sectional side view of a diagrammatic representation of a portion of an artery 900 through which a pulse 902 is propagating. The block arrow in FIG. 9 shows the direction of blood flow and pulse propagation. As diagrammatically shown, the propagating pulse 902 causes strain in the arterial walls 904, which is manifested in the form of an enlargement in the diameter (and consequently the cross-sectional area) of the arterial walls-referred to as “distension.” The spatial length L of an actual propagating pulse along an artery (along the direction of blood flow) is typically comparable to the length of a limb, such as the distance from a subject's shoulder to the subject's wrist or finger, and is generally less than one meter (m). However, the length/of a propagating pulse can vary considerably from subject to subject, and for a given subject, can vary significantly over durations of time depending on various factors. The spatial length L of a pulse will generally decrease with increasing distance from the heart until the pulse reaches capillaries.

As described above, some particular implementations relate to devices, systems and methods for estimating blood pressure or other cardiovascular characteristics based on estimates of an arterial distension waveform. The terms “estimating,” “measuring,” “calculating,” “inferring,” “deducing,” “evaluating,” “determining” and “monitoring” may be used interchangeably herein where appropriate unless otherwise indicated. Similarly, derivations from the roots of these terms also are used interchangeably where appropriate; for example, the terms “estimate,” “measurement,” “calculation,” “inference” and “determination” also are used interchangeably herein. In some implementations, the pulse wave velocity (PWV) of a propagating pulse may be estimated by measuring the pulse transit time (PTT) of the pulse as it propagates from a first physical location along an artery to another more distal second physical location along the artery. It will be appreciated that this PTT is different from the PTT that is described above with reference to FIG. 15. However, either version of the PTT may be used for the purpose of blood pressure estimation. Assuming that the physical distance ΔD between the first and the second physical locations is ascertainable, the PWV can be estimated as the quotient of the physical spatial distance ΔD traveled by the pulse divided by the time (PTT) the pulse takes in traversing the physical spatial distance ΔD. Generally, a first sensor positioned at the first physical location is used to determine a starting time (also referred to herein as a “first temporal location”) at which point the pulse arrives at or propagates through the first physical location. A second sensor at the second physical location is used to determine an ending time (also referred to herein as a “second temporal location”) at which point the pulse arrives at or propagates through the second physical location and continues through the remainder of the arterial branch. In such examples, the PTT represents the temporal distance (or time difference) between the first and the second temporal locations (the starting and the ending times).

The fact that measurements of the arterial distension waveform are performed at two different physical locations implies that the estimated PWV inevitably represents an average over the entire path distance ΔD through which the pulse propagates between the first physical location and the second physical location. More specifically, the PWV generally depends on a number of factors including the density of the blood p, the stiffness E of the arterial wall (or inversely the elasticity), the arterial diameter, the thickness of the arterial wall, and the blood pressure. Because both the arterial wall elasticity and baseline resting diameter (for example, the diameter at the end of the ventricular diastole period) vary significantly throughout the arterial system, PWV estimates obtained from PTT measurements are inherently average values (averaged over the entire path length ΔD between the two locations where the measurements are performed).

In traditional methods for obtaining PWV, the starting time of the pulse has been obtained at the heart using an electrocardiogram (ECG) sensor, which detects electrical signals from the heart. For example, the starting time can be estimated based on the QRS complex—an electrical signal characteristic of the electrical stimulation of the heart ventricles. In such approaches, the ending time of the pulse is typically obtained using a different sensor positioned at a second location (for example, a finger). As a person having ordinary skill in the art will appreciate, there are numerous arterial discontinuities, branches, and variations along the entire path length from the heart to the finger. The PWV can change by as much as or more than an order of magnitude along various stretches of the entire path length from the heart to the finger. As such, PWV estimates based on such long path lengths are unreliable.

In various implementations described herein, PTT estimates are obtained based on measurements (also referred to as “arterial distension data” or more generally as “sensor data”) associated with an arterial distension signal obtained by each of a first arterial distension sensor 906 and a second arterial distension sensor 908 proximate first and second physical locations, respectively, along an artery of interest. In some particular implementations, the first arterial distension sensor 906 and the second arterial distension sensor 908 are advantageously positioned proximate first and second physical locations between which arterial properties of the artery of interest, such as wall elasticity and diameter, can be considered or assumed to be relatively constant. In this way, the PWV calculated based on the PTT estimate is more representative of the actual PWV along the particular segment of the artery. In turn, the blood pressure P estimated based on the PWV is more representative of the true blood pressure. In some implementations, the magnitude of the distance ΔD of separation between the first arterial distension sensor 906 and the second arterial distension sensor 908 (and consequently the distance between the first and the second locations along the artery) can be in the range of about 1 centimeter (cm) to tens of centimeters-long enough to distinguish the arrival of the pulse at the first physical location from the arrival of the pulse at the second physical location, but close enough to provide sufficient assurance of arterial consistency. In some specific implementations, the distance ΔD between the first and the second arterial distension sensors 906 and 908 can be in the range of about 1 cm to about 30 cm, and in some implementations, less than or equal to about 20 cm, and in some implementations, less than or equal to about 10 cm, and in some specific implementations less than or equal to about 5 cm. In some other implementations, the distance ΔD between the first and the second arterial distension sensors 906 and 908 can be less than or equal to 1 cm, for example, about 0.1 cm, about 0.25 cm, about 0.5 cm or about 0.75 cm. By way of reference, a typical PWV can be about 15 meters per second (m/s). Using an ambulatory monitoring device in which the first and the second arterial distension sensors 906 and 908 are separated by a distance of about 5 cm, and assuming a PWV of about 15 m/s implies a PTT of approximately 3.3 milliseconds (ms).

The value of the magnitude of the distance ΔD between the first and the second arterial distension sensors 906 and 908, respectively, can be preprogrammed into a memory within a monitoring device that incorporates the sensors (for example, such as a memory of, or a memory configured for communication with, the control system 106 that is described above with reference to FIG. 1). As will be appreciated by a person of ordinary skill in the art, the spatial length L of a pulse can be greater than the distance ΔD from the first arterial distension sensor 906 to the second arterial distension sensor 908 in such implementations. As such, although the diagrammatic pulse 902 shown in FIG. 9 is shown as having a spatial length L comparable to the distance between the first arterial distension sensor 906 and the second arterial distension sensor 908, in actuality each pulse can typically have a spatial length L that is greater and even much greater than (for example, about an order of magnitude or more than) the distance ΔD between the first and the second arterial distension sensors 906 and 908.

In some implementations of the ambulatory monitoring devices disclosed herein, both the first arterial distension sensor 906 and the second arterial distension sensor 908 are sensors of the same sensor type. In some such implementations, the first arterial distension sensor 906 and the second arterial distension sensor 908 are identical sensors. In such implementations, each of the first arterial distension sensor 906 and the second arterial distension sensor 908 utilizes the same sensor technology with the same sensitivity to the arterial distension signal caused by the propagating pulses, and has the same time delays and sampling characteristics. In some implementations, each of the first arterial distension sensor 906 and the second arterial distension sensor 908 is configured for photoacoustic plethysmography (PAPG) sensing, e.g., as disclosed elsewhere herein. Some such implementations include a light source system and two or more ultrasonic receivers, which may be instances of the light source system 104 and the receiver system 102 of FIG. 1. In some implementations, each of the first arterial distension sensor 906 and the second arterial distension sensor 908 is configured for ultrasound sensing via the transmission of ultrasonic signals and the receipt of corresponding reflections. In some alternative implementations, each of the first arterial distension sensor 906 and the second arterial distension sensor 908 may be configured for impedance plethysmography (IPG) sensing, also referred to in biomedical contexts as bioimpedance sensing. In various implementations, whatever types of sensors are utilized, each of the first and the second arterial distension sensors 906 and 908 broadly functions to capture and provide arterial distension data indicative of an arterial distension signal resulting from the propagation of pulses through a portion of the artery proximate to which the respective sensor is positioned. For example, the arterial distension data can be provided from the sensor to a processor in the form of voltage signal generated or received by the sensor based on an ultrasonic signal or an impedance signal sensed by the respective sensor.

As described above, during the systolic phase of the cardiac cycle, as a pulse propagates through a particular location along an artery, the arterial walls expand according to the pulse waveform and the elastic properties of the arterial walls. Along with the expansion is a corresponding increase in the volume of blood at the particular location or region, and with the increase in volume of blood an associated change in one or more characteristics in the region. Conversely, during the diastolic phase of the cardiac cycle, the blood pressure in the arteries decreases and the arterial walls contract. Along with the contraction is a corresponding decrease in the volume of blood at the particular location, and with the decrease in volume of blood an associated change in the one or more characteristics in the region.

In the context of bioimpedance sensing (or impedance plethysmography), the blood in the arteries has a greater electrical conductivity than that of the surrounding or adjacent skin, muscle, fat, tendons, ligaments, bone, lymph, or other tissues. The susceptance (and thus the permittivity) of blood also is different from the susceptances (and permittivities) of the other types of surrounding or nearby tissues. As a pulse propagates through a particular location, the corresponding increase in the volume of blood results in an increase in the electrical conductivity at the particular location (and more generally an increase in the admittance, or equivalently a decrease in the impedance). Conversely, during the diastolic phase of the cardiac cycle, the corresponding decrease in the volume of blood results in an increase in the electrical resistivity at the particular location (and more generally an increase in the impedance, or equivalently a decrease in the admittance).

A bioimpedance sensor generally functions by applying an electrical excitation signal at an excitation carrier frequency to a region of interest via two or more input electrodes, and detecting an output signal (or output signals) via two or more output electrodes. In some more specific implementations, the electrical excitation signal is an electrical current signal injected into the region of interest via the input electrodes. In some such implementations, the output signal is a voltage signal representative of an electrical voltage response of the tissues in the region of interest to the applied excitation signal. The detected voltage response signal is influenced by the different, and in some instances time-varying, electrical properties of the various tissues through which the injected excitation current signal is passed. In some implementations in which the bioimpedance sensor is operable to monitor blood pressure, heartrate or other cardiovascular characteristics, the detected voltage response signal is amplitude- and phase-modulated by the time-varying impedance (or inversely the admittance) of the underlying arteries, which fluctuates synchronously with the user's heartbeat as described above. To determine various biological characteristics, information in the detected voltage response signal is generally demodulated from the excitation carrier frequency component using various analog or digital signal processing circuits, which can include both passive and active components.

In some examples incorporating ultrasound sensors, measurements of arterial distension may involve directing ultrasonic waves into a limb towards an artery, for example, via one or more ultrasound transducers. Such ultrasound sensors also are configured to receive reflected waves that are based, at least in part, on the directed waves. The reflected waves may include scattered waves, specularly reflected waves, or both scattered waves and specularly reflected waves. The reflected waves provide information about the arterial walls, and thus the arterial distension.

In some implementations, regardless of the type of sensors utilized for the first arterial distension sensor 906 and the second arterial distension sensor 908, both the first arterial distension sensor 906 and the second arterial distension sensor 908 can be arranged, assembled, or otherwise included within a single housing of a single ambulatory monitoring device. As described above, the housing and other components of the monitoring device can be configured such that when the monitoring device is affixed or otherwise physically coupled to a subject, both the first arterial distension sensor 906 and the second arterial distension sensor 908 are in contact with or in close proximity to the skin of the user at first and second locations, respectively, separated by a distance ΔD, and in some implementations, along a stretch of the artery between which various arterial properties can be assumed to be relatively constant. In various implementations, the housing of the ambulatory monitoring device is a wearable housing or is incorporated into or integrated with a wearable housing. In some specific implementations, the wearable housing includes (or is connected with) a physical coupling mechanism for removable non-invasive attachment to the user. The housing can be formed using any of a variety of suitable manufacturing processes, including injection molding and vacuum forming, among others. In addition, the housing can be made from any of a variety of suitable materials, including, but not limited to, plastic, metal, glass, rubber and ceramic, or combinations of these or other materials. In particular implementations, the housing and coupling mechanism enable full ambulatory use. In other words, some implementations of the wearable monitoring devices described herein are noninvasive, not physically-inhibiting and generally do not restrict the free uninhibited motion of a subject's arms or legs, enabling continuous or periodic monitoring of cardiovascular characteristics such as blood pressure even as the subject is mobile or otherwise engaged in a physical activity. As such, the ambulatory monitoring device facilitates and enables long-term wearing and monitoring (for example, over days, weeks, or a month or more without interruption) of one or more biological characteristics of interest to obtain a better picture of such characteristics over extended durations of time, and generally, a better picture of the user's health.

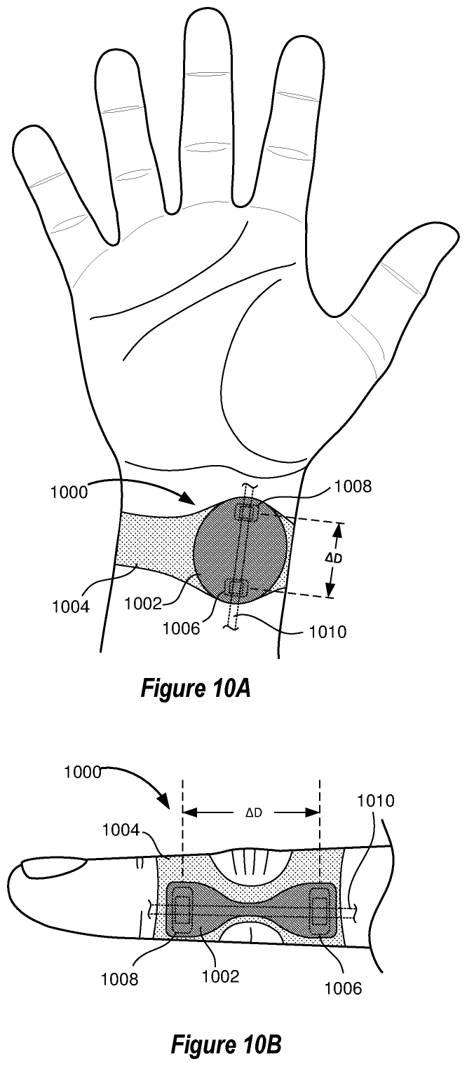

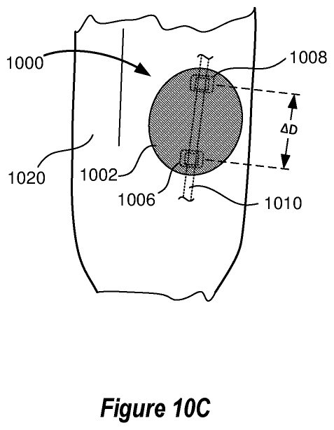

In some implementations, the ambulatory monitoring device can be positioned around a wrist of a user with a strap or band, similar to a watch or fitness/activity tracker. FIG. 10A shows an example ambulatory monitoring device 1000 designed to be worn around a wrist according to some implementations. In the illustrated example, the monitoring device 1000 includes a housing 1002 integrally formed with, coupled with, or otherwise integrated with a wristband 1004. The first and the second arterial distension sensors 1006 and 1008 may, in some instances, each include an instance of the ultrasonic receiver system 102 and a portion of the light source system 104 that are described above with reference to FIG. 1. In this example, the ambulatory monitoring device 1000 is coupled around the wrist such that the first and the second arterial distension sensors 1006 and 1008 within the housing 1002 are each positioned along a segment of the radial artery 1010 (note that the sensors are generally hidden from view from the external or outer surface of the housing facing the subject while the monitoring device is coupled with the subject, but exposed on an inner surface of the housing to enable the sensors to obtain measurements through the subject's skin from the underlying artery). Also as shown, the first and the second arterial distension sensors 1006 and 1008 are separated by a fixed distance ΔD. In some other implementations, the ambulatory monitoring device 1000 can similarly be designed or adapted for positioning around a forearm, an upper arm, an ankle, a lower leg, an upper leg, or a finger (all of which are hereinafter referred to as “limbs”) using a strap or band.

FIG. 10B shows an example ambulatory monitoring device 1000 designed to be worn on a finger according to some implementations. The first and the second arterial distension sensors 1006 and 1008 may, in some instances, each include an instance of the ultrasonic receiver 102 and a portion of the light source system 104 that are described above with reference to FIG. 1.

In some other implementations, the ambulatory monitoring devices disclosed herein can be positioned on a region of interest of the user without the use of a strap or band. For example, the first and the second arterial distension sensors 1006 and 1008 and other components of the monitoring device can be enclosed in a housing that is secured to the skin of a region of interest of the user using an adhesive or other suitable attachment mechanism (an example of a “patch” monitoring device).

FIG. 10C shows an example ambulatory monitoring device 1000 designed to reside on an earbud according to some implementations. According to this example, the ambulatory monitoring device 1000 is coupled to the housing of an earbud 1020. The first and second arterial distension sensors 1006 and 1008 may, in some instances, each include an instance of the ultrasonic receiver 102 and a portion of the light source system 104 that are described above with reference to FIG. 1.

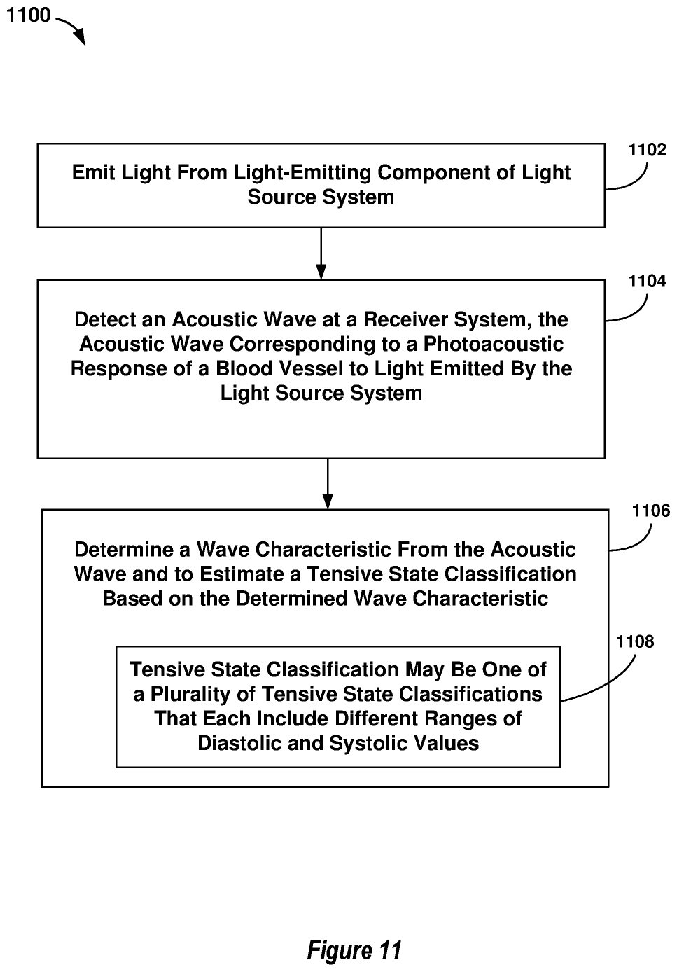

FIG. 11 is a flow diagram that shows examples of some disclosed operations. The blocks of FIG. 11 may, for example, be performed by the apparatus 100 of FIG. 1 or by a similar apparatus. For example, the processes of FIG. 11 may be performed by the control system 106. As with other methods disclosed herein, the method outlined in FIG. 11 may include more or fewer processes than indicated. Moreover, the blocks of methods disclosed herein are not necessarily performed in the order indicated. In some instances, one or more of the blocks shown in FIG. 11 may be performed concurrently or alternatively.

At 1102, a light source system may emit light from a light-emitting component. For example, the light emitting component 608 of the light source system 602 of FIG. 6 may illuminate human or other animal tissue.

At 1104, a receiver system configured to detect an acoustic wave corresponding to a photoacoustic response of a blood vessel to light emitted by the light source system. For instance, the receiver system 604 of FIG. 6 may receive the photoacoustic response of the blood vessel 616 in the form of the acoustic wave signal 610.

At 1106, a control system may determine a wave characteristic from the acoustic wave and to estimate a tensive state classification based on the determined wave characteristic. For example, the control system 606 of FIG. 6 may determine the wave characteristic 614 from the acoustic wave 610 and to estimate the tensive state classification 612. The tensive state classification may be one of a plurality of tensive state classifications at 1108 that each include different ranges of diastolic and systolic values.

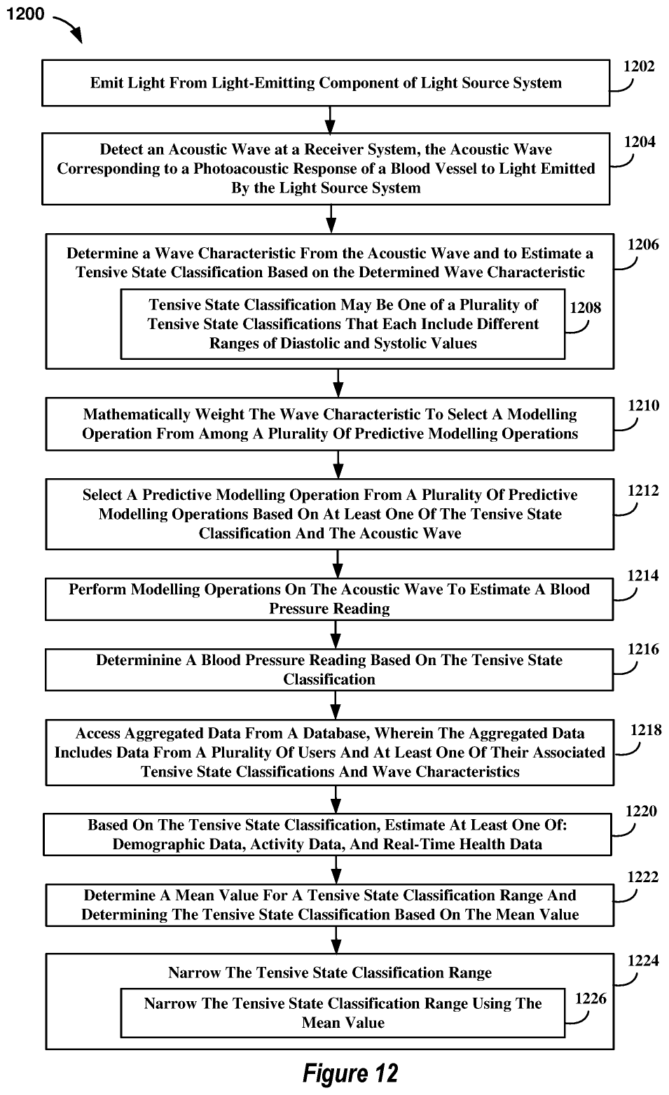

FIG. 12 is a flow diagram that shows additional examples of some disclosed operations. The illustrative method 1200 generally includes receiving and inputting characteristics of a photoacoustic signal to a classifier. An implementation of the method may input data from the characteristics of the photoacoustic signal to a machine learning regression learning model to predict blood pressure. Alternatively or additionally, the tensive state classification estimate may be used by the classifier to select a most effective model based on the wave characteristics of the photoacoustic signal. The blocks of FIG. 12 may, for example, be performed by the apparatus 100 of FIG. 1 or by a similar apparatus. For example, the processes of FIG. 12 may be performed by the control system 106. As with other methods disclosed herein, the method outlined in FIG. 12 may include more or fewer blocks than indicated. Moreover, the blocks of methods disclosed herein are not necessarily performed in the order indicated. In some instances, one or more of the blocks shown in FIG. 12 may be performed concurrently or alternatively.

Turning more particularly to the flow diagram, a light source system may at 1202 emit light from a light-emitting component. For example, the light emitting component 608 of the light source system 602 of FIG. 6 may illuminate arterial tissue.

At 1204, a receiver system configured to detect an acoustic wave corresponding to a photoacoustic response of a blood vessel to light emitted by the light source system. For instance, the receiver system 604 of FIG. 6 may receive the photoacoustic response of the blood vessel 616 in the form of the acoustic wave signal 610.

At 1206, a control system may determine a wave characteristic from the acoustic wave and to estimate a tensive state classification based on the determined wave characteristic. For example, the control system 606 of FIG. 6 may determine the wave characteristic 614 from the acoustic wave 610 and to estimate the tensive state classification 612. The tensive state classification may be one of a plurality of tensive state classifications at 1208 that each include different ranges of diastolic and systolic values.

According to a particular implementation, the method 1200 at 1210 may include mathematically weighting the wave characteristic to select a modelling operation from among a plurality of predictive modelling operations. For instance, the control system 404b of FIG. 4 may execute the weighting algorithm 440 to mathematically weight one or more characteristics of the acoustic wave 410. The control system 404b may use the weighted scheme to select one or more predictor models 416.

Additionally or alternatively at 1212, the method 1200 may include selecting a predictive modelling operation from a plurality of predictive modelling operations based on at least one of the tensive state classification and the acoustic wave. For example, the control system 404b of FIG. 4 may select one or more predictor models 416 based on at least one of the characteristics of the acoustic wave signal 410 or the tensive state classifications 408.

At 1214, an implementation of the method 1200 may include performing modelling operations on the acoustic wave to estimate a blood pressure reading. For instance, the control system 404b of FIG. 4 may execute one or more of the predictor models 416 to estimate a blood pressure estimate output 406.

The illustrative method 1200 may include estimating at 1216 a blood pressure reading based on the tensive state classification. Continuing with the example of FIG. 4, the control system 404b may estimate a blood pressure estimate output 406 at 1216 a blood pressure reading based on the tensive state classifications 408.

At 1218, an implementation of the method 1200 may include accessing aggregated data from a database, where the aggregated data includes data from a plurality of users and at least one of their associated tensive state classifications and wave characteristics. For instance, the control system 404b of FIG. 4 may access the aggregated data 430. The aggregated data 430 may include at least one of the tensive state classifications 408 and the characteristics of the acoustic wave signal 410.

At 1220, the method 1200 may include, based on the tensive state classification, estimating at least one of: demographic data, activity data, and real-time health data. For example, the control system 404b of FIG. 4 may estimate at least one of demographic information 432, activity information 436, and real-time health data output, such as blood pressure estimate outputs 406.

The illustrative method 1200 may at 1222 include determining a mean value for a tensive state classification range and determining the tensive state classification based on the mean value. For example, the control system 404b of FIG. 4 may execute the mean error predictor module 446 to determine a mean value and tensive classification range to determine the tensive state classifications 1208.

At 1224, the method 1200 may include narrowing the tensive state classification range. Further, the method 1200 at 1226 include narrowing the tensive state classification range using the mean value. For example, the For example, the control system 404b of FIG. 4 may execute the mean error predictor module 446 and use the determined mean value to narrow one or more of the tensive state classifications 1208.

Implementation examples are described in the following numbered clauses:

1. An apparatus, comprising:
a light source system including a light-emitting component;
a receiver system configured to detect an acoustic wave corresponding to a photoacoustic response of a blood vessel to light emitted by the light source system; and
a control system configured to determine a wave characteristic from the acoustic wave and to estimate a tensive state classification based on the determined wave characteristic, wherein the tensive state classification is one of a plurality of tensive state classifications that each include different ranges of diastolic and systolic values.

2. The apparatus of clause 1, wherein the control system is further configured to determine a blood pressure reading based on the tensive state classification.

3. The apparatus of clauses 1 and 2, wherein the control system is further configured, based on the tensive state classification, to estimate at least one of: demographic data, activity data, and real-time health data.

4. The apparatus of clauses 1-3, wherein the control system is further configured to perform modelling operations on the acoustic wave to estimate a blood pressure reading.

5. The apparatus of clauses 1-4, wherein the control system is further configured to select a predictive modelling operation from a plurality of predictive modelling operations based on at least one of the tensive state classification and the acoustic wave.

6. The apparatus of clauses 1-5, wherein the control system is further configured to mathematically weight the wave characteristic to select a modelling operation from among a plurality of predictive modelling operations.

7. The apparatus of clauses 1-6, wherein the control system is further configured to access aggregated data from a database, wherein the aggregated data includes data from a plurality of users and at least one of their associated tensive state classifications and wave characteristics.

8. The apparatus of clauses 1-7, wherein the control system is further configured to determine a mean value for a tensive state classification range and determine the tensive state classification based on the mean value.

9. The apparatus of clauses 1-8, wherein the control system is further configured to narrow a tensive state classification range.

10. A method of using a photoacoustic signal to classify a tensive state of a user, the method comprising:
detecting an acoustic wave corresponding to a photoacoustic response of a blood vessel to light emitted by a light source system; and
determining a wave characteristic from the acoustic wave and estimating a tensive state classification based on the determined wave characteristic, wherein the tensive state classification is one of a plurality of tensive state classifications that each include different ranges of diastolic and systolic values.

11. The method of clause 10, further comprising determining a blood pressure reading based on the tensive state classification.

12. The method of clauses 10 and 11, further comprising, based on the tensive state classification, estimating at least one of: demographic data, activity data, and real-time health data.

13. The method of clauses 10-12, further comprising performing modelling operations on the acoustic wave to estimate a blood pressure reading.

14. The method of clauses 10-13, further comprising selecting a predictive modelling operation from a plurality of predictive modelling operations based on at least one of the tensive state classification and the acoustic wave.

15. The method of clauses 10-14, further comprising mathematically weighting the wave characteristic to select a modelling operation from among a plurality of predictive modelling operations.

16. The method of clauses 10-15, further comprising accessing aggregated data from a database, wherein the aggregated data includes data from a plurality of users and at least one of their associated tensive state classifications and wave characteristics.

17. The method of clauses 10-16, further comprising determining a mean value for a tensive state classification range and determining the tensive state classification based on the mean value.

18. The method of clauses 10-17, further comprising narrowing the tensive state classification range.

19. The method of clauses 10-18, further comprising narrowing the tensive state classification range using the mean value.

20. An apparatus comprising:
a means for detecting an acoustic wave corresponding to a photoacoustic response of a blood vessel to light emitted by a light source system; and
a means for determining a wave characteristic from the acoustic wave and estimating a tensive state classification based on the determined wave characteristic, wherein the tensive state classification is one of a plurality of tensive state classifications that each include different ranges of diastolic and systolic values.

21. The apparatus of clause 20, wherein the means for determining the wave characteristic is further configured to determine a blood pressure reading based on the tensive state classification.

22. The apparatus of clauses 20 and 21, wherein the means for determining the wave characteristic is further configured, based on the tensive state classification, to estimate at least one of: demographic data, activity data, and real-time health data.

23. The apparatus of clauses 20-22, wherein the means for determining the wave characteristic is further configured to perform modelling operations on the acoustic wave to estimate a blood pressure reading.

24. The apparatus of clauses 20-23, wherein the means for determining the wave characteristic is further configured to select a predictive modelling operation from a plurality of predictive modelling operations based on at least one of the tensive state classification and the acoustic wave.

25. The apparatus of clauses 20-24, wherein the means for determining the wave characteristic is further configured to mathematically weight the wave characteristic to select a modelling operation from among a plurality of predictive modelling operations.

26. The apparatus of clauses 20-25, wherein the means for determining the wave characteristic is further configured to access aggregated data from a database, wherein the aggregated data includes data from a plurality of users and at least one of their associated tensive state classifications and wave characteristics.

27. The apparatus of clauses 20-26, wherein the means for determining the wave characteristic is further configured to determine a mean value for a tensive state classification range and determine the tensive state classification based on the mean value.

28. A computer-readable medium storing computer executable code for classifying a tensive state of a user, the computer executable code being configured to:
detect an acoustic wave corresponding to a photoacoustic response of a blood vessel to light emitted by a light source system; and
determine a wave characteristic from the acoustic wave and estimate a tensive state classification based on the determined wave characteristic, wherein the tensive state classification is one of a plurality of tensive state classifications that each include different ranges of diastolic and systolic values.

29. The computer-readable medium of clause 28, wherein the computer executable code is further configured to, based on the tensive state classification, estimate at least one of: demographic data, activity data, and real-time health data.

30. The computer-readable medium of clauses 28 and 29, wherein the computer executable code is further configured to perform modelling operations on the acoustic wave to estimate a blood pressure reading.

As used herein, a phrase referring to “at least one of” a list of items refers to any combination of those items, including single members. As an example, “at least one of: a, b, and c” is intended to cover: a, b, c, a-b, a-c, b-c, and a-b-c.

The various illustrative logics, logical blocks, modules, circuits, and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, or, any conventional processor, controller, microcontroller, or state machine. A processor also may be implemented as a combination of computing devices, e.g., a combination of a DSP and a microprocessor, a plurality of microprocessors, one or more microprocessors in conjunction with a DSP core, or any other such configuration. In some implementations, particular processes and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also may be implemented as one or more computer programs, i.e., one or more modules of computer program instructions, encoded on a computer storage media for execution by, or to control the operation of, data processing apparatus.

If implemented in software, the functions may be stored on or transmitted over as one or more instructions or code on a computer-readable medium, such as a non-transitory medium. The processes of a method or algorithm disclosed herein may be implemented in a processor-executable software module which may reside on a computer-readable medium. Computer-readable media include both computer storage media and communication media including any medium that may be enabled to transfer a computer program from one place to another. Storage media may be any available media that may be accessed by a computer. By way of example, and not limitation, non-transitory media may include RAM, ROM, EEPROM, CD-ROM or other optical disk storage, magnetic disk storage or other magnetic storage devices, or any other medium that may be used to store desired program code in the form of instructions or data structures and that may be accessed by a computer. Also, any connection may be properly termed a computer-readable medium. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those having ordinary skill in the art, and the generic principles defined herein may be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “exemplary” is used exclusively herein, if at all, to mean “serving as an example, instance, or illustration.” Any implementation described herein as “exemplary” is not necessarily to be construed as preferred or advantageous over other implementations.

Certain features that are described in this specification in the context of separate implementations also may be implemented in combination in a single implementation. Conversely, various features that are described in the context of a single implementation also may be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products. Additionally, other implementations are within the scope of the following claims. In some cases, the actions recited in the claims may be performed in a different order and still achieve desirable results.

It will be understood that unless features in any of the particular described implementations are expressly identified as incompatible with one another or the surrounding context implies that they are mutually exclusive and not readily combinable in a complementary and/or supportive sense, the totality of this disclosure contemplates and envisions that specific features of those complementary implementations may be selectively combined to provide one or more comprehensive, but slightly different, technical solutions. It will therefore be further appreciated that the above description has been given by way of example only and that modifications in detail may be made within the scope of this disclosure.

Various modifications to the implementations described in this disclosure may be readily apparent to those skilled in the art, and the generic principles defined herein may be applied to other implementations without departing from the spirit or scope of this disclosure. Thus, the following claims are not intended to be limited to the implementations shown herein, but are to be accorded the widest scope consistent with this disclosure, the principles and the novel features disclosed herein.

Additionally, certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flow diagram. However, other operations that are not depicted can be incorporated in the example processes that are schematically illustrated. For example, one or more additional operations can be performed before, after, simultaneously, or between any of the illustrated operations. Moreover, various ones of the described and illustrated operations can itself include and collectively refer to a number of sub-operations. For example, each of the operations described above can itself involve the execution of a process or algorithm. Furthermore, various ones of the described and illustrated operations can be combined or performed in parallel in some implementations. Similarly, the separation of various system components in the implementations described above should not be understood as requiring such separation in all implementations. As such, other implementations are within the scope of the following claims. In some cases, the actions recited in the claims can be performed in a different order and still achieve desirable results.

What is claimed is:

1. An apparatus, comprising:
a light source system including a light-emitting component;
a receiver system configured to detect an acoustic wave corresponding to a photoacoustic response of a blood vessel to light emitted by the light source system; and
a control system configured to determine a wave characteristic from the acoustic wave and to estimate a tensive state classification based on the determined wave characteristic, wherein the tensive state classification is one of a plurality of tensive state classifications that each include different ranges of diastolic and systolic values.

2. The apparatus of claim 1, wherein the control system is further configured to determine a blood pressure reading based on the tensive state classification.

3. The apparatus of claim 1, wherein the control system is further configured, based on the tensive state classification, to estimate at least one of: demographic data, activity data, and real-time health data.

4. The apparatus of claim 1, wherein the control system is further configured to perform modelling operations on the acoustic wave to estimate a blood pressure reading.

5. The apparatus of claim 1, wherein the control system is further configured to select a predictive modelling operation from a plurality of predictive modelling operations based on at least one of the tensive state classification and the acoustic wave.

6. The apparatus of claim 1, wherein the control system is further configured to mathematically weight the wave characteristic to select a modelling operation from among a plurality of predictive modelling operations.

7. The apparatus of claim 1, wherein the control system is further configured to access aggregated data from a database, wherein the aggregated data includes data from a plurality of users and at least one of their associated tensive state classifications and wave characteristics.

8. The apparatus of claim 1, wherein the control system is further configured to determine a mean value for a tensive state classification range and determine the tensive state classification based on the mean value.

9. The apparatus of claim 1, wherein the control system is further configured to narrow a tensive state classification range.

10. A method of using a photoacoustic signal to classify a tensive state of a user, the method comprising:
detecting an acoustic wave corresponding to a photoacoustic response of a blood vessel to light emitted by a light source system; and
determining a wave characteristic from the acoustic wave and estimating a tensive state classification based on the determined wave characteristic, wherein the tensive state classification is one of a plurality of tensive state classifications that each include different ranges of diastolic and systolic values.

11. The method of claim 10, further comprising determining a blood pressure reading based on the tensive state classification.

12. The method of claim 10, further comprising, based on the tensive state classification, estimating at least one of: demographic data, activity data, and real-time health data.

13. The method of claim 10, further comprising performing modelling operations on the acoustic wave to estimate a blood pressure reading.

14. The method of claim 10, further comprising selecting a predictive modelling operation from a plurality of predictive modelling operations based on at least one of the tensive state classification and the acoustic wave.

15. The method of claim 10, further comprising mathematically weighting the wave characteristic to select a modelling operation from among a plurality of predictive modelling operations.

16. The method of claim 10, further comprising accessing aggregated data from a database, wherein the aggregated data includes data from a plurality of users and at least one of their associated tensive state classifications and wave characteristics.

17. The method of claim 10, further comprising determining a mean value for a tensive state classification range and determining the tensive state classification based on the mean value.

18. The method of claim 17, further comprising narrowing the tensive state classification range.

19. The method of claim 17, further comprising narrowing the tensive state classification range using the mean value.

20. An apparatus comprising:
a means for detecting an acoustic wave corresponding to a photoacoustic response of a blood vessel to light emitted by a light source system; and
a means for determining a wave characteristic from the acoustic wave and estimating a tensive state classification based on the determined wave characteristic, wherein the tensive state classification is one of a plurality of tensive state classifications that each include different ranges of diastolic and systolic values.

21. The apparatus of claim 20, wherein the means for determining the wave characteristic is further configured to determine a blood pressure reading based on the tensive state classification.

22. The apparatus of claim 20, wherein the means for determining the wave characteristic is further configured, based on the tensive state classification, to estimate at least one of: demographic data, activity data, and real-time health data.

23. The apparatus of claim 20, wherein the means for determining the wave characteristic is further configured to perform modelling operations on the acoustic wave to estimate a blood pressure reading.

24. The apparatus of claim 20, wherein the means for determining the wave characteristic is further configured to select a predictive modelling operation from a plurality of predictive modelling operations based on at least one of the tensive state classification and the acoustic wave.

25. The apparatus of claim 20, wherein the means for determining the wave characteristic is further configured to mathematically weight the wave characteristic to select a modelling operation from among a plurality of predictive modelling operations.

26. The apparatus of claim 20, wherein the means for determining the wave characteristic is further configured to access aggregated data from a database, wherein the aggregated data includes data from a plurality of users and at least one of their associated tensive state classifications and wave characteristics.

27. The apparatus of claim 20, wherein the means for determining the wave characteristic is further configured to determine a mean value for a tensive state classification range and determine the tensive state classification based on the mean value.

28. A computer-readable medium storing computer executable code for classifying a tensive state of a user, the computer executable code being configured to:
detect an acoustic wave corresponding to a photoacoustic response of a blood vessel to light emitted by a light source system; and
determine a wave characteristic from the acoustic wave and estimate a tensive state classification based on the determined wave characteristic, wherein the tensive state classification is one of a plurality of tensive state classifications that each include different ranges of diastolic and systolic values.

29. The computer-readable medium of claim 28, wherein the computer executable code is further configured to, based on the tensive state classification, estimating at least one of: demographic data, activity data, and real-time health data.

30. The computer-readable medium of claim 28, wherein the computer executable code is further configured to perform modelling operations on the acoustic wave to estimate a blood pressure reading.
